(19) United States
(12) Patent Application Publication
(10) Pub. No.: US 2021/0282632 A1
(43) Pub. Date: Sep. 16, 2021

(54) VIDEO TOOTHBRUSH

(71) Applicant: SHENZHEN WILLSEA E-COMMERCE CO. LIMITED, Shenzhen (CN)

(72) Inventors: Wentao Hu, Shenzhen (CN); Zhenjie Mo, Shenzhen (CN)

(73) Assignee: SHENZHEN WILLSEA E-COMMERCE CO. LIMITED, Shenzhen (CN)

(21) Appl. No.: 17/235,933

(22) Filed: Apr. 20, 2021

Related U.S. Application Data
(63) Continuation-in-part of application No. 16/848,794, filed on Apr. 14, 2020, now Pat. No. 10,980,410.
Application No. 16/848,794 is a continuation of application No. PCT/CN2020/076651, filed on Feb. 25, 2020.

(51) Int. Cl. A61B 1/24; A61C 17/34; A46B 15/00; A46B 9/04; A46B 5/00; A46B 13/02

(52) U.S. Cl. CPC A61B 1/24; A61C 17/3481; A46B 15/0055; A46B 9/04; A61C 17/3436; A46B 15/0022; A46B 13/023; A61C 17/3445; A46B 5/0095

(57) ABSTRACT

Embodiments of the disclosure provide a video toothbrush. An exemplary video toothbrush may include a handle housing an exciter and a support element including a support neck and a support head. The support element may be configured to extend from a proximal connection with the handle to a distal end. The video toothbrush may further include a camera mounted near the distal end of the support head and a shaft extending from the support neck and configured to support a cleaning head toward a distal end of the shaft and configured to receive vibration from the exciter toward a proximal end of the shaft. The video toothbrush may also include the cleaning head having a plurality of bristles projecting in a first direction. The cleaning head may define a central opening. The central opening may be arranged to provide the camera with a view in the first direction.

### CROSS-REFERENCE TO RELATED APPLICATION

The present application is a continuation-in-part of U.S. application Ser. No. 16/848,794, filed on Apr. 14, 2020, which is a bypass continuation of International Patent Application No. PCT/CN2020/076651, filed on Feb. 25, 2020. The entire contents of each of the above-identified applications are expressly incorporated herein by reference.

### TECHNICAL FIELD

The present disclosure relates to systems and methods for a video toothbrush, such as a toothbrush with a video camera aligned with bristles or other cleaning elements and configured to provide a live video output for brushing quality enhancement or other purposes.

### BACKGROUND

The field of toothbrushes has been filled with innovations since the 1400s. In the last half century, electric toothbrushes have been introduced. Today, toothbrushes continue to incorporate further technological innovations.

Recently there have been attempts to incorporate video into electric toothbrushes to provide video electric toothbrushes. These attempts have been mainly unsuccessful for a variety of reasons. For example, certain toothbrushes have incorporated video as a secondary feature, such that a user can take a video image of the user's own teeth at a first time, and brush the user's teeth at a second time. Other approaches may permit video images of the user's teeth during brushing, but can only do so from an odd or confusing angle.

Embodiments of the disclosure address the above problems by providing improved video toothbrush systems, which may have a video camera aligned with bristles or other cleaning elements and configured to provide a live video output for brushing quality enhancement and improved user experience. Furthermore, certain embodiments of the disclosure reduce the mouth impact of the video toothbrush, permitting ease of simultaneous brushing and video observation, as well as ease of insertion into the mouth of the user.

### SUMMARY

Embodiments of the disclosure provide an apparatus that may include a handle housing an exciter. The apparatus may also include a support element including a support neck and a support head. The support element may be configured to extend from a proximal connection with the handle to a distal end. The apparatus may further include a camera mounted near the distal end of the support head. The apparatus may additionally include a shaft extending from the support neck and configured to support a cleaning head toward a distal end of the shaft and configured to receive vibration from the exciter toward a proximal end of the shaft The apparatus may also include the cleaning head having a plurality of bristles projecting in a first direction. The cleaning head may define a central opening. The central opening may be arranged to provide the camera with a view in the first direction.

Embodiments of the disclosure also provide a further apparatus that may include a handle with an exciter. The apparatus may also include a support element including a support neck and a support head. The support element may be configured to extend from a proximal connection with the handle to a distal end. The apparatus may further include a camera mounted near the distal end of the support head. The apparatus may additionally include a shaft extending from the support neck and configured to support a cleaning head toward a distal end of the shaft and configured to receive vibration from the exciter toward a proximal end of the shaft. The camera may be configured to view through a central opening of the cleaning head upon installation of the cleaning head.

Embodiments of the disclosure also provide a further apparatus that may include a handle comprising an exciter and an elongated support head extending from a proximal connection with the handle to a distal end. The elongated support head supports a camera mounted near the distal end of the support head. The apparatus also includes a cleaning head extending in parallel with the elongated support head. The cleaning head includes a bristle portion at a distal end of the cleaning head. The cleaning head is connected to the exciter by a shaft configured to transmit kinetic energy from the exciter to the cleaning head, at least a portion of the shaft being enclosed by the cleaning head. The bristle portion includes a plurality of bristles arranged to expose an opening on the cleaning head, the opening providing the camera with a view in a first direction.

Embodiments of the disclosure additionally provide a further apparatus that may include a handle that may include an exciter and a support head that may extend from a proximal connection with the handle to a distal end of the support head. The support head supporting a camera and comprising mounting elements for a cleaning head having a plurality of bristles, the mounting elements being configured to removably mount the cleaning head to the support head near the distal end of the support head. The apparatus also includes a shaft extending from the exciter to the distal end of the support head, the shaft being configured to transmit kinetic energy from the exciter to the cleaning head through the mounting elements. The camera is configured to provide a view in a first direction through an opening of the cleaning head after the cleaning head is installed onto the support head through the mounting elements

Embodiments of the disclosure also provide a further apparatus that may include a handle that may include an exciter and a support head that may extend from a proximal connection with the handle to a distal end. The support head may support a camera at the distal end of the support head and may include a shaft that may extend from the exciter to the distal end of the support head. The shaft may be configured to support a cleaning head that may include a plurality of bristles at a distal end of the shaft and may be configured to transmit kinetic energy from the exciter to the cleaning head. The camera may be positioned on the support head between the cleaning head and the handle.

Embodiments of the disclosure additionally provide a further apparatus that may include a handle that may include an exciter and a support head that may extend from a proximal connection with the handle to a distal end. The support head may support a camera at the distal end of the support head. The apparatus may also include a shaft extending from the exciter to the distal end of the support head. The shaft may be configured to support a cleaning head including a plurality of bristles at a distal end of the shaft and may be configured to transmit kinetic energy from the exciter to the cleaning head. The shaft may be at least partially enclosed by the cleaning head and the support head, and the cleaning head may include an elongated portion that extends from a portion of the cleaning head including the plurality of bristles. The elongated portion may attach the cleaning head to the support head.

Embodiments of the disclosure also provide a further apparatus that may include a handle including an exciter. The apparatus may also include an elongated support head extending from a proximal connection with the handle to a distal end having a bumpy rear portion. The elongated support head may support a camera mounted near the distal end of the elongated support head. The apparatus may also include a cleaning head extending in parallel with the elongated support head. The cleaning head may include a bristle portion that may include a plurality of bristles at a distal end of the cleaning head on a front side of the cleaning head. The cleaning head may be connected to the exciter by a shaft configured to transmit kinetic energy from the exciter to the cleaning head, where at least a portion of the shaft may be enclosed by the cleaning head. The bumpy rear portion may include a plurality of bumps that may be arranged to expose an opening on the elongated support head. The opening may provide the camera with a view in a direction towards a rear side of the elongated support head.

It is to be understood that both the foregoing general description and the following detailed description are exemplary and explanatory only and are not restrictive of the invention, as claimed.

### BRIEF DESCRIPTION OF THE DRAWINGS

FIG. 1 illustrates a perspective view of a video toothbrush, according to certain embodiments.

FIG. 2 illustrates a vertical cross-section of a video toothbrush, according to certain embodiments.

FIG. 3 illustrates a side view of a video toothbrush, according to certain embodiments.

FIG. 4 illustrates a perspective view of a video toothbrush without a cleaning head, according to certain embodiments.

FIG. 5 illustrates a video toothbrush with bumpers, according to certain embodiments.

FIG. 6 illustrates a functional block diagram of a handle of a video toothbrush handle, according to certain embodiments.

FIG. 7 illustrates a method of operation of an exemplary video toothbrush, according to certain embodiments.

FIG. 8 illustrates a video toothbrush with a rotating cleaning head, according to certain embodiments.

FIG. 9 illustrates a perspective view of a video toothbrush with a rotating cleaning head, according to certain embodiments.

FIG. 10 illustrates a side view of a video toothbrush, according to certain embodiments.

FIG. 11 illustrates a vertical cross-section of a video toothbrush, according to certain embodiments.

FIG. 12 illustrates perspective views of a video toothbrush, according to certain embodiments.

FIG. 13 illustrates perspective views of a video toothbrush, according to certain embodiments.

FIG. 14 illustrates a perspective view of a video toothbrush, according to certain embodiments.

FIG. 15 illustrates a perspective view of a video toothbrush, according to certain embodiments.

FIG. 16 illustrates perspective views of a video toothbrush, according to certain embodiments.

FIG. 17 illustrates a perspective view of a video toothbrush with a detachable cleaning head, according to certain embodiments.

FIG. 18 illustrates a perspective view of a video toothbrush with a detachable cleaning head, according to certain embodiments.

FIG. 19 illustrates a perspective view of a video toothbrush, according to certain embodiments.

FIG. 20 illustrates a perspective view of a video toothbrush and a paired display, according to certain embodiments.

### DETAILED DESCRIPTION

Certain embodiments of the present disclosure provide a video toothbrush. For example, certain embodiments provide a toothbrush that can obtain video of dental cleaning during the cleaning process. In an example, a camera can be provided behind vibrating bristles, which may permit the simultaneous cleaning of teeth and monitoring of the surface status of the teeth being cleaned. Certain embodiments provide vibration isolation or dampening, which may improve the quality of video captured by the toothbrush or reduce the performance requirements of an embedded camera or processor.

For example, a camera with a frame capture speed sufficiently fast may be unaffected by vibrations of the toothbrush. Nevertheless, such high-speed cameras may have other limitations, such as cost, complexity, and/or bulk. Likewise, vibration interference may be resolved by suitable image processing techniques, such as image stabilization. Nevertheless, to perform such stabilization in real time in the presence of a highly unstable camera may require significant computational power, which may lead to additional cost, complexity, and/or bulk.

The camera of certain embodiments may be co-axially aligned with a brushing head and have a clearance for isolating vibration. The clearance may be sufficiently narrow to reduce the bulkiness and permit easy handling of the toothbrush.

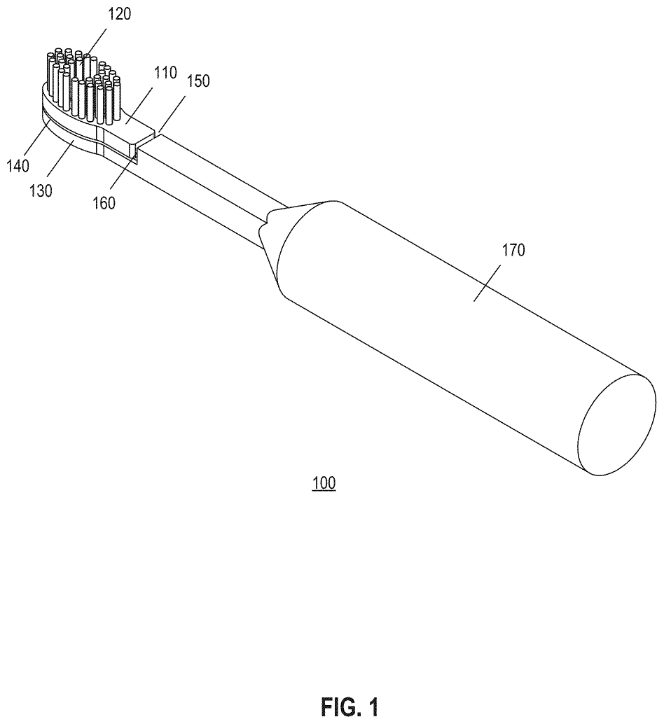

FIG. 1 illustrates a perspective view of a video toothbrush according to certain embodiments. As shown in FIG. 1, a device 100 can include a handle 170 and a cleaning head 110 with bristles 120. Device 100 can also include a support head 130 separated from the cleaning head 110 by first clearance 140 in the vertical direction and separated from the cleaning head 110 by second clearance 150 in the horizontal direction. First clearance 140 may be between 0.5 mm to 5 mm. For example, first clearance 140 may be 0.5 mm, 1 mm, 1.5 mm, 2 mm, 2.5 mm, 3 mm, 3.5 mm, 4 mm, 4.5 mm, 5 mm, or within any range defined by the above-listed values. Second clearance 150 may be between 0.5 mm to 5 mm. For example, second clearance 150 may be 0.5 mm, 1 mm, 1.5 mm, 2 mm, 2.5 mm, 3 mm, 3.5 mm, 4 mm, 4.5 mm, 5 mm, or within any range defined by the above-listed values. In certain embodiments, the direction toward the cleaning head 110 may be considered the distal direction, while the direction toward handle 170 may be considered the proximal direction.

Cleaning head 110 may be supported by shaft 160, which may extend along the horizontal direction inside support head 130. Shaft 160, which is obscured from the angle shown in FIG. 1 but passes through second clearance 150, may be configured to transfer vibrations from a motor inside handle 170 to the cleaning head 110. Shaft 160 may also be configured to support cleaning head 110 when bristles 120 are pressed against teeth (not shown). A camera, not visible in this view, may be provided in support head 130. Wires for the camera may run inside support head 130 and connect to processors, transmitters, receivers, and the like, inside handle 170.

Handle 170 may be sealed and waterproofed. For example, handle 170 may be configured to resist water according to ingress protection (IP) code 7, which may indicate that harmful quantities of water cannot enter the enclosure when the enclosure is immersed in water of up to 1 meter depth for at least a few minutes. In some embodiments, higher IP ratings (e.g., IP code 8) or lower IP ratings (e.g., IP code 6) may be used, depending on the requirements of particular applications. Other levels of water resistance or water proofing are also permitted. The IP codes are published by the International Electrotechnical Commission (IEC) and described at IEC standard 60529 with a corresponding European standard EN 60529.

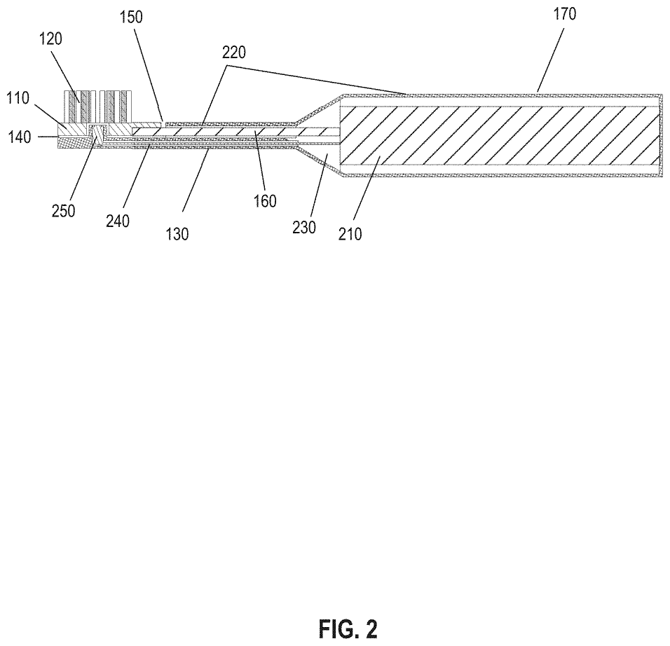

FIG. 2 illustrates a vertical cross-section of a toothbrush according to certain embodiments. As shown in FIG. 2, shaft 160 may link motor 210 to cleaning head 110. Handle 170 can be formed from an outer housing 220, annular spacer 230. and a compartment for motor 210. The compartment housing motor 210 may also include (not shown) a control circuit board, a wireless transmission module, one or more battery, or the like. Also not shown, the housing 220 may include one or more port, such as an electric power port for charging and/or a universal serial bus (USB) port for wired communication and/or charging. In some embodiments, a coil may be provided for wireless charging. Other ports are also permitted.

Cabling 240 may connect the compartment housing motor 210 to a camera 250. Cabling 240 can include power and signaling wires. The camera 250 may be provided with an illumination source, such as one or more light emitting diodes (LEDs). In FIG. 2, camera 250 and LEDs are shown as a single bundle, but they may be scattered. For example, multiple LEDs may be arranged in a circular pattern around the periphery of an end of the toothbrush, while camera 250 itself may be centered. Nevertheless, in certain embodiments, the LEDs may be located immediately adjacent to and in approximately the same plane as a lens of camera 250.

In certain embodiments, the camera may be located in handle 170 and fiber optics may be provided so that the camera can view directly out of the support head 130 through the fiber optics.

Cleaning head 110 may be separable from or integral with shaft 160. Likewise, shaft 160 may be permanently installed or may be removable. Thus, for example, in certain embodiments the clean head 110 may be removable/replaceable or both the cleaning head 110 and the shaft 160 may be removable/replaceable.

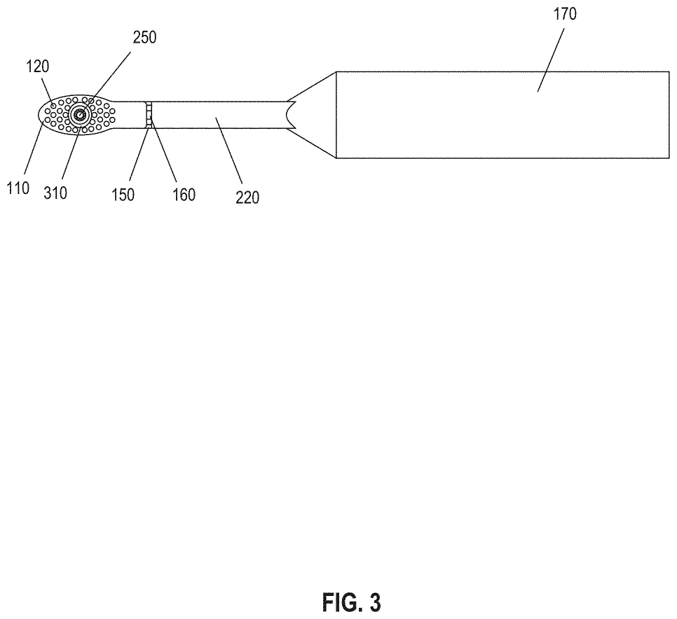

FIG. 3 illustrates a side view of a video toothbrush according to certain embodiments. The view of FIG. 3 may be orthogonal to the view of FIG. 2. As shown in FIG. 3, the housing 220 can extend from the handle 170. Second clearance 150 can separate the cleaning head 110 from the housing 220, while shaft 160 can bridge second clearance 150 and provide vibrational motion to the cleaning head 110 and bristles 120.

As also shown in FIG. 3, a third clearance 310 may be an annular gap between a casing of camera 250 and cleaning head 110. Third clearance 310 may be obtained by providing a circular opening in cleaning head 110. In certain embodiments, the shape of third clearance 310 may be adjusted to an oval or rounded rectangle, to permit clearance to be present as the cleaning head 110 reciprocates in the left-right direction.

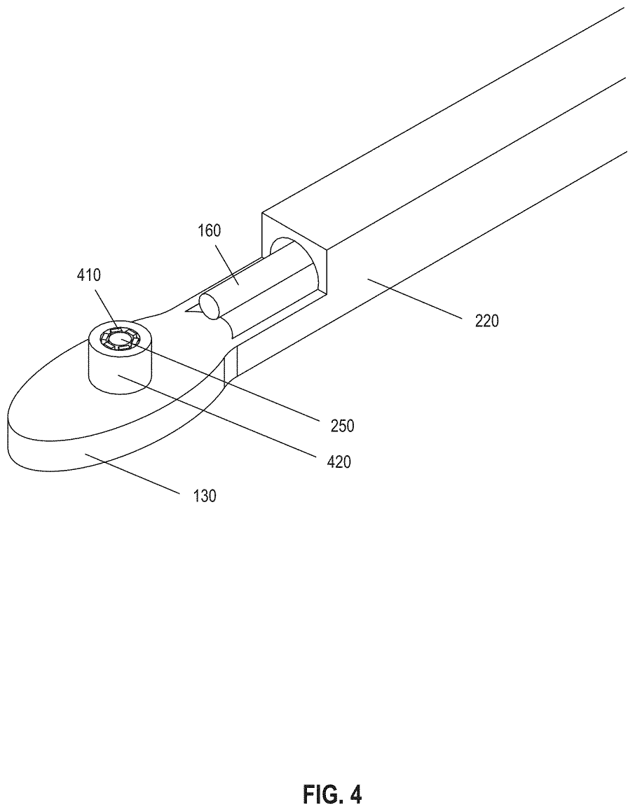

FIG. 4 illustrates a perspective view of a video toothbrush without a cleaning head, according to certain embodiments. As mentioned above, in certain embodiments the cleaning head may be removed. FIG. 4 illustrates such an embodiment. In the embodiment of FIG. 4, the shaft 160 has not been removed. An end of shaft 160 may be a simple cylinder as shown, or may be provided with a strip, a detent mechanism, threads, a ball or socket, or other mechanism configured to permit non-destructive attachment and removal of the cleaning head.

As shown in FIG. 4, camera 250 can be surrounded by adjacent LEDs 410. In this case, six LEDs 410 are shown, although other numbers of LEDs 410 are permitted. The LEDs 410 may emit light having any desired wavelength. In certain embodiments, blue or ultraviolet (UV) LEDs 410 may be used to provide illumination of dental surfaces and/or to help highlight a contrast agent such as a plaque disclosing tablet. An example of such a disclosing agent is Erythrosine, which may appear black under blue light.

As also shown in FIG. 4. camera 250 and LEDs 410 may be mounted inside a casing 420. Casing 420 can be integral with support head 130, which may also be integral with housing 220. Thus, for example, casing 420, support head 130, and housing 220 may be formed as a single continuous piece.

In FIG. 4, camera 250 is illustrated as being mounted at about the same level as shaft 160. Other arrangements are also possible, such as mounting the camera at a position higher than shaft 160 (and consequently closer to target teeth of a user) or at a position lower than shaft 160 (and consequently farther from the target teeth).

Shaft 160 is shown as extending approximately through a central axis of housing 220, although other arrangements are also possible.

The arrangement of FIGS. 1 through 4 may be viewed of as an example of a floating cleaning head embodiment. In this approach, the cleaning head would have no or only slight contact with camera casing, the support head, or the housing of the handle under regular use scenarios. For example, shaft 160 and cleaning head 110 may be sufficiently rigid such that clearance 140 can be substantially maintained during the cleaning process. In another example, the back surface of cleaning head 110 and/or the front surface of support head 130 that defines clearance 140 may be coated with a low-friction material to allow free or low-friction movement of cleaning head 110 relative to support head 130 even when they contact each other under external pressure. Further modifications are also possible.

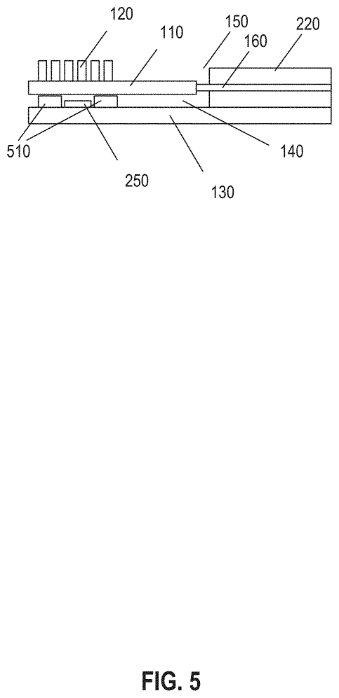

FIG. 5 illustrates a video toothbrush with bumpers according to certain embodiments. As shown in FIG. 5, cleaning head 110 can be separated from housing 220 by second clearance 150 and separated from support head 130 by first clearance 140. In addition, one or more bumpers 510 can be provided. Bumpers 510 may be provided as a pair of lines or a ring around camera 250. Bumpers 510 may be configured to prevent cleaning head 110 from being deflected into contact with camera 250 during use. For example, as bristles 120 press against a user's teeth, the cleaning head 110 may be forced in a downward direction toward support head 130. Bumpers 510 may limit motion of the cleaning head 110 in this direction. Bumpers 510 can be formed from a material that limits transmission of vibration between the cleaning head 110 and support head 130.

In some embodiments, a video toothbrush can be provided with a port configured to allow one or more battery to be inserted or removed. The video toothbrush can also be provided with a communication module that is configured to permit transfer of images from the camera to a display remote from the video toothbrush. For example, a USB port may be used to transfer video data over a wired connection from the video toothbrush to a smart phone. Similarly, Bluetooth and/or wireless network (e.g., WiFi) communication equipment, including an antenna, may be included and may be configured to wirelessly transmit video data to a smart phone or other display.

In the above discussion a motor is identified as a possible exciter to transmit motion through a shaft to the cleaning head. Other exciters are also possible, including machines that convert hand pumping into vibrational motion, or piezoelectric translators/transducers.

The camera can be configured with one or more lenses. An outermost lens may be coated with an anti-fog surface treatment, such as a layer comprising titanium dioxide. In certain embodiments, the anti-fog capabilities of the titanium dioxide may be enhanced by the use of at least one UV LED. Other hydrophilic layers configured to limit fogging may also be used. The lens may be configured to provide a desired image and may be a macro lens, a fish-eye lens, or any other suitable lens. For example, the lens may have a short focus length (e.g., a macro lens) to be able to focus on the surface of the teeth that is about 1 centimeter or less in distance from the lens. In another example, the lens may have a wide field of view to cover a relatively large area of the teeth being cleaned. In certain embodiments, two or more cameras may be employed in a side-by-side configuration to provide stereoscopic images.

In some embodiments, additional vibration remediation techniques can be employed. For example, the support head may include vibration dampening materials or vibration dampening structures, such as mechanical joints, rubber, foam, or combinations thereof. For example, the annular spacer 230 may be made from a material that dampens vibrations.

In some embodiments, external ports may be provided to permit charging of the device. Alternatively or additionally, battery compartment doors may be provided to permit replacement of the batteries. In certain embodiments, batteries and/or super capacitors may be configured for wireless recharging. For example, the video toothbrush may be provided with a stand that permits air drying of the bristles and wireless charging of the toothbrush.

The various views of the above-described embodiment do not explicitly show a power button. In certain embodiments a power button may be provided on the handle of the video toothbrush. In some embodiments, activation of the video toothbrush may be performed by a remote device, such as a smart phone.

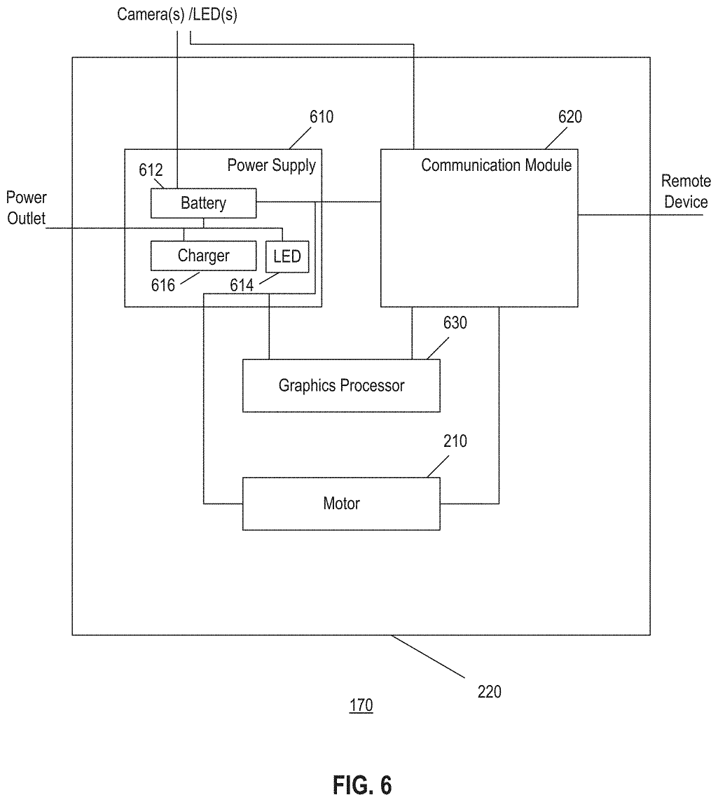

FIG. 6 illustrates a functional block diagram of handle 170 of a video toothbrush, according to certain embodiments. As shown in FIG. 6, housing 220 may house not only motor 210, but also other components. For example, a power supply 610 can be provided including a battery 612, status LED 614, and charger 616. The power supply 610 can be connected to an external power outlet and can supply power to the camera(s)/LED(s) of the head of the toothbrush. The power supply 610 can also power a communication module 620, graphics processor 630, and a motor 210 within the housing. The communication module 620 can communicate data with the camera(s)/LED(s) of the head of the toothbrush, can provide instructions to motor 210 of the toothbrush, and can communicate data and control signals with a remote device, such as a smart phone. The communication module 620 may thus provide a communication interface to the remote device over which commands, data, and the like may be provided. The graphics processor 630 may be used for video image acquisition and/or processing, such as image stabilization, image compression, or other features. Other elements may also be included within the housing including, for example, memory, a timer, a speaker for providing an audible alarm or instruction, a user interface display, buttons, or the like. Additional sensors can be provided, include motion sensors, ranging sensors, and the like. The motion sensors and/or ranging sensors may be used to aid in determination of a current position of the video toothbrush. Sensor data can be passed to the remote device together with camera data.

In some embodiments, the video toothbrush may include a toothpaste dispenser configured to deliver toothpaste to the bristles. This dispenser may be refillable or may be a disposable portion of the replaceable cleaning head. In some embodiments, the toothpaste may be foamless to aid video capture.

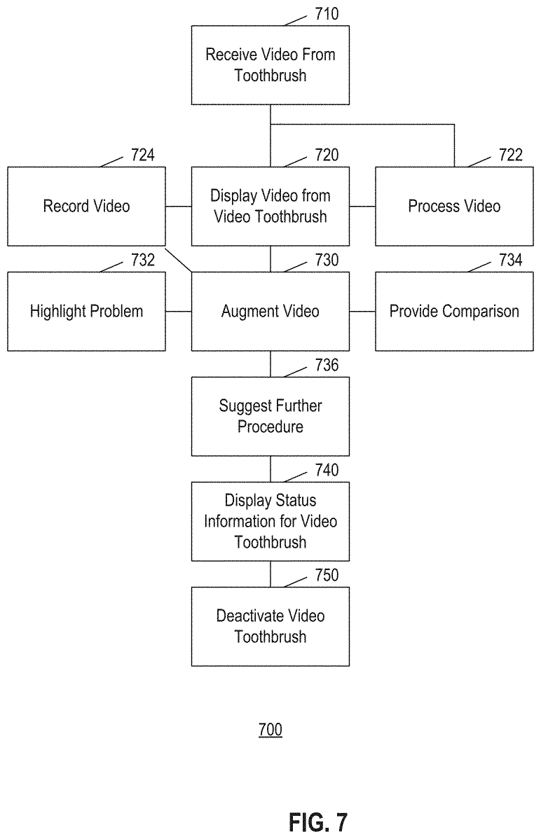

FIG. 7 illustrates a method 700 according to certain embodiments. Method 700 may be performed by, for example, a smart phone or other device remote from the video toothbrush. For example, a display installed in or behind a bathroom mirror surface could be used. Certain steps of method 700 may also be performed, at least partially, by toothbrush 100. Method 700 may include a plurality of steps, as shown in FIG. 7. In some embodiments, one or more steps shown in FIG. 7 may be omitted, or one or more additional steps may be added to method 700. The steps of method 700 may be performed in different order from that shown in FIG. 7, and some steps may be performed simultaneously.

As shown in FIG. 7, method 700 can begin, at step 710, with receiving video from a video toothbrush (e.g., video toothbrush 100). The video toothbrush may be activated to capture and transmit video signals. This may be performed via a user pressing a button or moving a switch on the video toothbrush. In some embodiments, the activation can be performed using a smart phone or other remote device. In certain embodiments, activating the video toothbrush can involve activating movement of the bristles only, activating video only, or activating both movement and video. After activation, video of the surface of teeth can be captured by, for example, camera 250, processed by graphics processor 630, and transmitted by communication module 620. A remote device such as a smart phone may receive the video signals via wired or wireless connections to the video toothbrush through, for example, communication module 620.

At step 720, video received from the video toothbrush can be provided on a display, such as on the screen of a smart phone.

In some embodiments, prior to displaying the video at step 720, a device may process the video at step 722. This video processing may be performed in the video toothbrush, at a remote device, such as a smartphone, or distributed between the video toothbrush and a remote device. The video processing may include aspects such as image stabilization or other enhancements.

Method 700 can also include, at step 730, augmenting the video. For example, at step 732, the method can include highlighting a problem. To highlight a problem, the remote device and/or the video toothbrush (hereinafter referred to as a system) may analyze a received video image for the presence of plaque or debris. The system may then highlight the presence of such plaque or debris, so that the video toothbrush can be controlled to address the highlighted issue. At step 734, the system may provide a comparison video or comparison still image. This may include a reference, such as an historical image or video, or a tutorial image or video. For example, the system can record video at step 724 and subsequently use the recorded video to augment live video at step 730 by providing a comparison at step 734. Motion and/or ranging sensors may be used to permit comparison between a currently captured image and an historical image. The comparison at step 734 may include a comparison of whiteness of teeth, for example by comparing an average whiteness of recent images of a tooth with average whiteness of historical images of the tooth.

Method 700 may also include, at step 736, suggesting a further procedure. For example, the system can analyze the video and determine areas that have not been cleaned yet or that have not been adequately cleaned. Thus, the system may provide a text-based suggestion, such as, “Don't forget to brush your molars!” or may provide a visual guide, such as pointing to an area that requires cleaning. In case the video toothbrush is being controlled robotically, the system can instruct the robot where to clean next, rather than merely providing a suggestion.

Method 700 may also include, at step 740, displaying status information for the video toothbrush. The status information may include information such as a current charge level of the video toothbrush, a connectivity quality between the video toothbrush and the remote device, any fault conditions experienced by the video toothbrush, a current number of uses or hours of usage of a given cleaning head, or a level of toothpaste in a dispenser. The system may be configured to prompt reordering or automatically reorder cleaning head(s) and/or toothpaste for a user of the video toothbrush.

At step 750, method 700 can include deactivating the video toothbrush. Such deactivation can involve placing the video toothbrush in a low power mode, such that it can be re-activated remotely, or completely powering off the video toothbrush.

Embodiments of the present disclosure may provide certain benefits and/or advantages. For example, certain embodiments may permit a user to look where the user is brushing, from approximately the same vantage point as the bristles. This may permit the user to accurately ascertain whether all tooth surfaces are being adequately and thoroughly cleaned, which may promote oral health and/or hygiene.

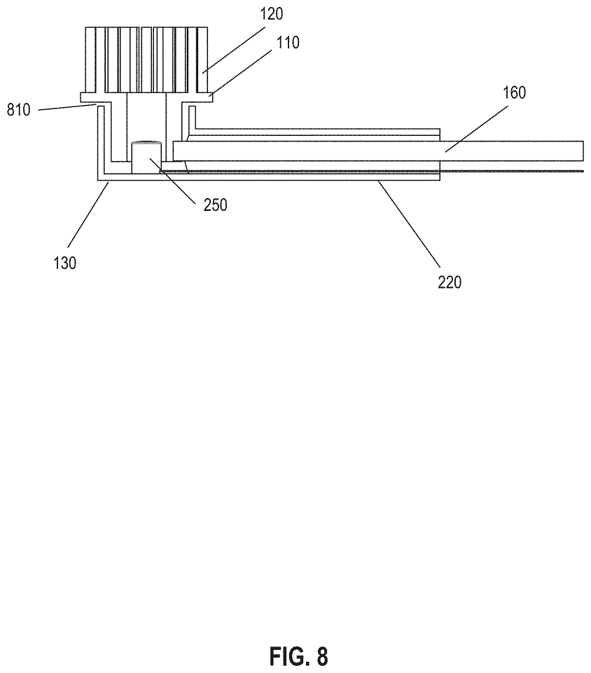

FIG. 8 illustrates a video toothbrush with a rotating cleaning head, according to certain embodiments. The embodiment of FIG. 8 may have many homologous structures to those shown in FIGS. 1-6. Accordingly, where it is convenient for ease of reference the same reference numbers may be used.

In the embodiment shown in FIGS. 1-5, it is assumed that the cleaning head 110 may move or translate in an approximately linear reciprocating motion. By contrast, in FIG. 8, the cleaning head 110 may move in a continuous or reciprocating rotational direction. In both embodiments, the cleaning head 110 may have a central open area through which camera 250 may view. In both embodiments, the camera 250 may be affixed to a support head 130, which may be integral with a housing 220. In both embodiments, a shaft 160 may transmit energy from an exciter, such as a motor, in a handle of the video toothbrush.

In the embodiment of FIG. 8. there may be an outer clearance 810 between the cleaning head 110 and the housing 220. Additionally, there may be bearings or similar features within the end of the video toothbrush, as the cleaning head 110 may comprise a roughly top-hot shaped member, with a cylindrical wall and a disk shaped brim from which the bristles 120 extend. The bearings, if used, may help to support and separate the cleaning head 110 from the housing 220. The housing 220 at the cleaning end of the video toothbrush may function as the support head 130.

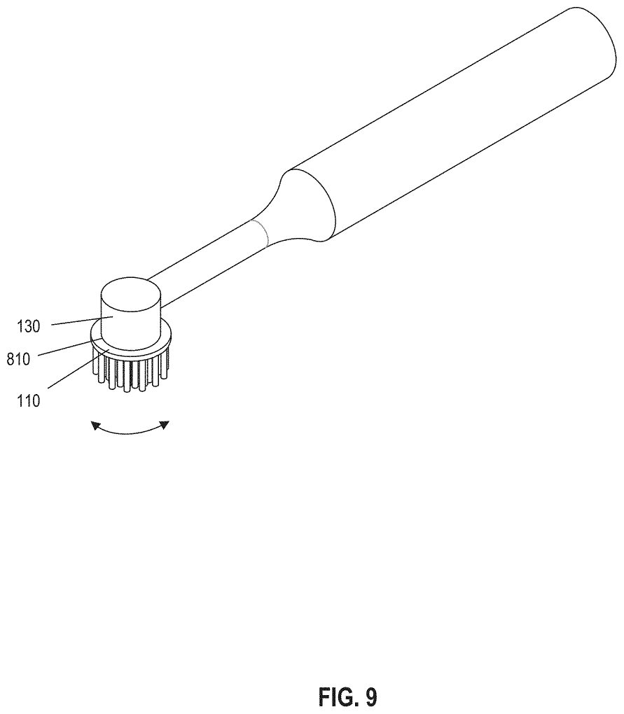

FIG. 9 illustrates a video toothbrush with a rotating cleaning head, according to certain embodiments. FIG. 9 can be considered as a perspective view of the embodiment shown in FIG. 8. As shown in FIG. 9, the cleaning head 110 can rotate, while the support head 130 may remain static and separated by clearance 810.

In the embodiments shown in FIGS. 8 and 9, clearance 810 may be small (for example, less than 5 mm, 2 mm, 1 mm, etc.), and cleaning head 110 may be configured to glide along the surface of support head 130 if contact is made between cleaning head 110 and support head 130.

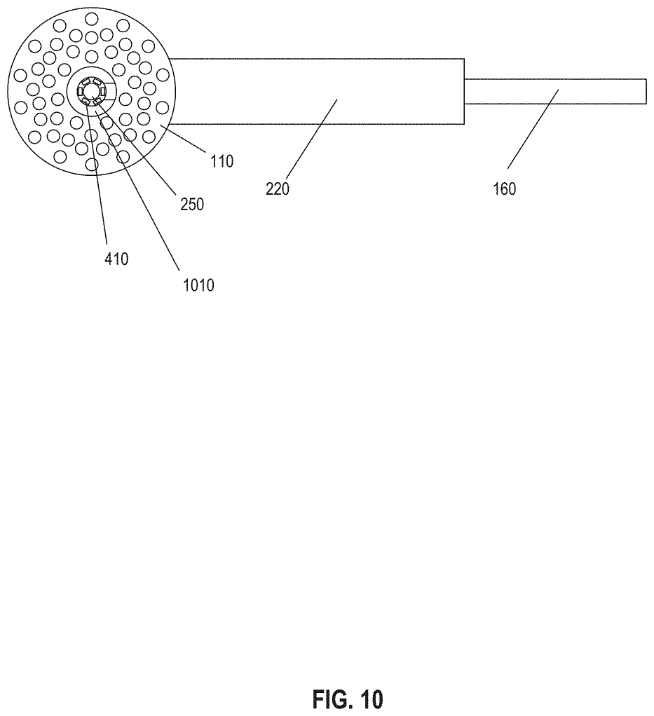

FIG. 10 illustrates a side view of a video toothbrush according to certain embodiments. The embodiment of FIG. 10 can be considered as another view of the same embodiment as FIG. 8 and FIG. 9. In the view provided by FIG. 10, a portion of housing 220 is shown cut away to reveal shaft 160 within. As can be seen from FIG. 10, an inner clearance 1010, analogous to third clearance 310 in FIG. 3, can be provided between a casing of camera 250 and cleaning head 110. The inner clearance 1010 may be approximately circular.

FIG. 10 also shows a plurality of LEDs 410, which may serve to illuminate the field of view of camera 250. The same variations and modifications discussed above with reference to FIG. 4 may similarly be applied with respect to the embodiment illustrated in FIG. 10.

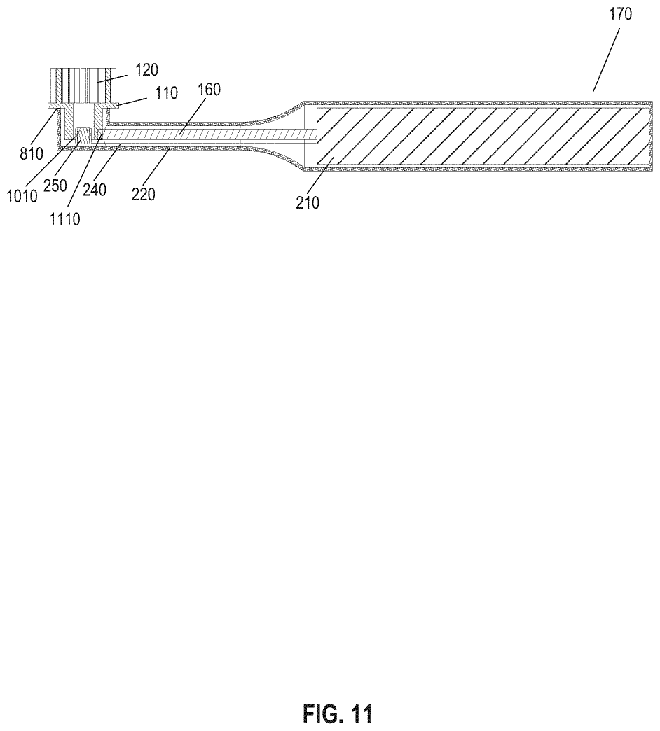

FIG. 11 illustrates a vertical cross-section of a video toothbrush according to certain embodiments. The embodiment shown in FIG. 11 may be considered a more complete embodiment similar to those shown in FIGS. 8-10. As shown in FIG. 11, the video toothbrush may include a handle 170.

The handle 170 may include a compartment for motor 210. As in the previously discussed embodiments, shaft 160 may extend from motor 210 to cleaning head 110. In this case, the shaft 160 may provide rotational motion and may transfer rotation to the cleaning head 110 using, for example, gearing at intersection 1110. The rotational motion may be a continuous rotational motion or a reciprocating rotational motion. A camera 250 may be connected with cabling 240, which may be flexible or inflexible power and signal wires.

Inner clearance 1010 can separate the casing of camera 250 from touching the cleaning head 110. Bristles 120 can extend from the surface of cleaning head 110. As can be seen in FIG. 11, outer clearance 810 may be nearly zero, such that the cleaning head 110 can slide or glide over housing 220.

As shown, the camera 250 may be significantly below the level of bristles 120. In other embodiments, the camera may extend higher, such that the camera may, for example, be level with the surface of cleaning head 110.

The handle of the embodiments of FIGS. 1-5 may be similar to the handle of the embodiments of FIGS. 8-11. Thus, the same discussion and modifications mentioned with reference to FIG. 6 may similarly apply to the embodiments of FIGS. 1-5 and 8-11. Likewise, the method shown and described with reference to FIG. 7 may be implemented using the systems shown in FIGS. 1-5 and 8-11.

Other embodiments are also possible. For example, the bristles may be attached to a static portion of the video toothbrush and a plate with holes to accommodate the bristles may be used to move the bristles. In this case, the plate with holes may be referred to as a cleaning head, even though the bristles are the elements actively cleaning teeth. Numerous other modifications and combinations thereof may be applied without departing from the above-identified principles.

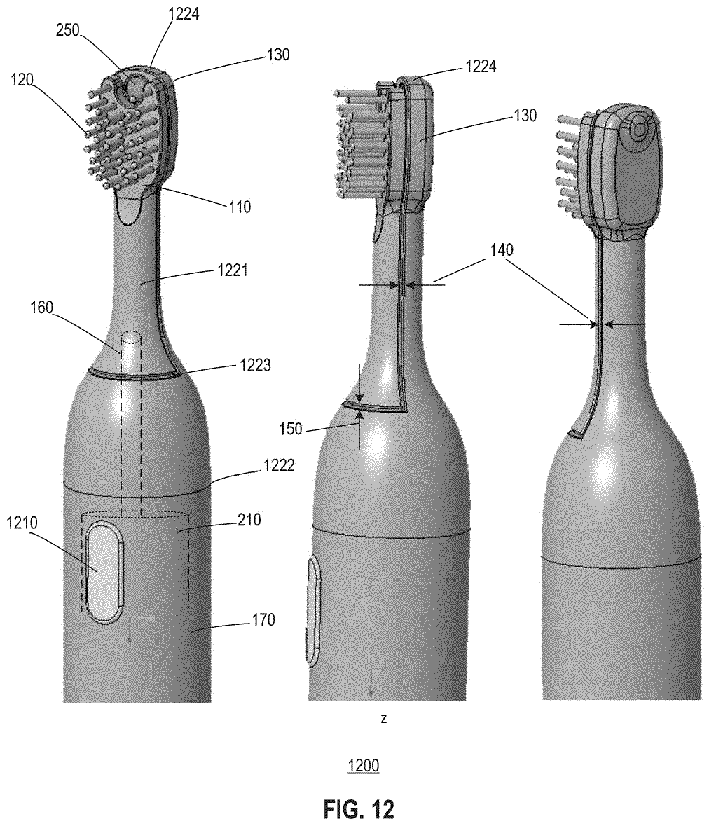

FIG. 12 illustrates perspective views of another video toothbrush 1200, according to certain embodiments. The three views show video toothbrush 1200 from three different angles. As shown in FIG. 12, cleaning head 110 of video toothbrush 1200 has a “neck” 1221 that is parallel to the support head 130, which in FIG. 12 takes the form of an elongated support head 130 that parallels the cleaning head 110.

Hereinafter, in drawings where it is appropriate, support head 130 is also referred to as elongated support head 130, reflecting the elongated shape extending from a proximal connection 1222 with handle 170 to a distal end 1224. However, it is noted that in some embodiments support head 130 may refer to an end portion mounted on or as an extension of handle 170 where the end portion does not have an elongated shape facing or opposite to the “neck” portion 1221 of cleaning head 110.

In FIG. 12, the elongated support head 130 includes an end portion (e.g., portion close to distal end 1224) that contains the camera 250 and face the portion of the cleaning head 110 with the bristles 120. The elongated support head 130 may also include an elongated portion between the interface (e.g., 1222) of the elongated support head 130 and the handle 170 and the end portion of the elongated support head 130. Such an elongated portion face the “neck” portion of the cleaning head 110. The shaft 160 used for transmitting kinetic energy to the cleaning head 110 is at least partially enclosed by the cleaning head 110.

For example, FIG. 12 shows a video toothbrush including a handle 170 comprising an exciter 210 such as motor 210 (e.g., similar to the embodiment shown in FIG. 2, included inside the handle 170). Additional aspects of the motor 210 are discussed elsewhere in the disclosure (e.g., with respect to FIG. 2). FIG. 12 also shows an elongated support head 130 extending from a proximal connection 1222 with the handle 170 to a distal end 1224. The elongated support head 130 supports a camera 250 mounted near the distal end 1224 of the support head 130. Further, the elongated support head 130 faces cleaning head 110 extending in parallel with the elongated support head 130.

For example, the handle 170 may be a cylindrical structure designed to be held by a user of the video toothbrush, such as when the user brushes his or her teeth using the video toothbrush. However, the handle 170 is not limited to a cylinder, and the handle 170 may take on other forms, such as a rectangular prism. The handle 170 may also include other features that deviate from a purely cylindrical shape. For example, the handle 170 may include indentations or other regions that make it easier for a user to firmly hold and manipulate the handle 170 while brushing his or her teeth.

The cleaning head 110 may include a bristle portion 120 including a plurality of bristles 120 at a distal end of the cleaning head 110. The cleaning head 110 is connected to the exciter 210 by a shaft 160 configured to transmit kinetic energy from the exciter 210 to the cleaning head 110, where at least a portion of the shaft 160 is enclosed by the cleaning head 110. The bristle portion 120 includes a plurality of bristles 120 arranged to expose an opening on the cleaning head 110, the opening providing the camera 250 with a view in a first direction, for example, in the direction parallel to the bristles 120, or perpendicular to the surface of support head 130 that faces cleaning head 110.

Shaft 160 may rotate or reciprocate to transmit the kinetic energy to the bristle portion 120 of cleaning head 110 to move the bristles 120. The kinetic energy may cause the bristles 120 to vibrate, rotate, or otherwise move to facilitate cleaning a user's teeth when used to brush the user's teeth.

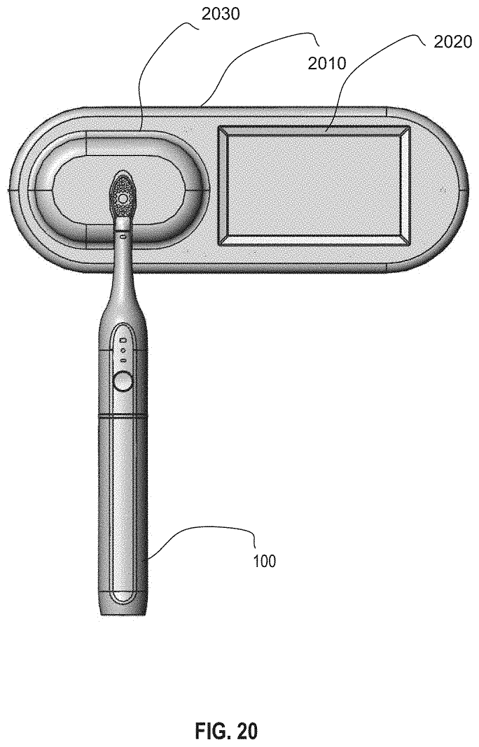

Video toothbrush 1200 may also include a battery compartment (e.g., included inside handle 170, not shown in FIG. 12) configured to hold a battery 612, where the battery 612 is electrically coupled to the motor 210 and serves as an energy source for the motor 210, as explained above in connection with FIG. 6. The battery 612 may also be electrically coupled to the camera 250 and provide an energy source for the camera 250. For example, the camera 250 may use energy when capturing images and/or video, or the camera 250 may require energy for functions such as performing a focusing adjustment. Also, as noted elsewhere, other sources of energy differing from a battery 612 are also possible. As will be discussed further, below, such as in the discussion of FIG. 20, the battery 612 may also provide electrical energy for wireless communications and/or computerized processing provided by the video toothbrush, as well as other functions of the video toothbrush.

The video toothbrush may also include at least one power button 1210 or power switch 1210. For example, the power button 1210 may be a button 1210 which, when pressed, toggles the video toothbrush between “on” and “off” states. However, the video toothbrush may also include a switch 1210 including an “on” state and an “off” state, where the switch 1210 includes two discrete positions.

The video toothbrush may have a single “on” and “off” state, such that when the video toothbrush is “on” both the camera 250 and the motor 210 operate, and when the video toothbrush is “off” both the camera 250 and the motor are inactive. Similarly, other functions of the video toothbrush may all be turned “on” or “off” together.

The video toothbrush may also include separate power buttons 1210 or switches 1210 for the camera 250 and the motor 210. In such embodiments, it is possible to use the camera 250 without activating the motor 210, and it is possible to use the motor 210 without activating the camera 250. Also, other functions of the video toothbrush may be controlled separately. For example, the wireless communications functions of the video toothbrush may be controlled remotely, such as by operating wireless communications upon receiving an activating signal, rather than by controls at the video toothbrush itself.

Furthermore, there may be additional control buttons or other control elements built into the video toothbrush. These elements may be buttons or toggle switches, as discussed above, but these are only non-limiting examples, and other examples of interacting with the toothbrush are possible. For example, twisting the elongated support head 130 where it extends from the handle 170 may be used as a way of affecting a zoom level or focus setting of the camera 250.

Thus, in FIG. 12, there is an elongated support head 130 that extends from a proximal connection 1222 with the handle 170 to a distal end 1224, the elongated support head 130 supporting a camera 250 mounted near the distal end 1224 of the support head 130. For example, the elongated support head 130 may extend from the handle 170, taper, and provide a flat region of the elongated support head 130 facing cleaning head 110.

FIG. 12 also shows an example where the interface between the elongated support head 130 and the handle 170 is a circular top portion of the handle 170. In the example of FIG. 12, the elongated support head 130 tapers before extending as a neck, at the end of which a camera 250 is integrated. The portions of the elongated support head 130 closer to handle 170 acts as a base for receiving the cleaning head 110.

In FIG. 12, the elongated support head 130 provides an area into which the cleaning head 110 fits. For example, a neck region of the cleaning head 110 parallels a neck region of the elongated support head 130, and a top portion of the cleaning head 110 includes bristles 120. As discussed, at the top of cleaning head 110, there may be an opening (e.g., in the form of a curved gap) to provide the camera 250 a view towards the bristle portion 120 from the top portion of the elongated support head 130.

However, having a circular portion between the top of handle 170 and the bottom of the receipt region for the cleaning head 110 is only an example, and it will be recognized that varying shapes and varying types of tapering may be present in various embodiments. For example, in other examples, the bottom of the elongated support head 130 may directly interface with a top portion of the handle 170, and the bottom of the cleaning head 110 directly contacts a corresponding top portion of the handle 170, rather than providing an intermediate portion of the elongated support head 130 between the handle 170 and a bottom of the “neck” of the cleaning head 110.

In some embodiments, the elongated support head 130 extends parallel to a central axis of the handle 170. However, in other embodiments, it is possible to have an elongated support head 130 that is inclined with respect to the handle 170, or even has a variable or modifiable tilt and/or curvature. The elongated support head 130 includes a camera 250 mounted in the elongated support head 130.

FIG. 12 shows embodiments where camera 250 is mounted at the top of the elongated support head 130. In such an example, there are at least two possible variations. FIG. 12 shows a particular example in which the top of the elongated support head 130 includes a rounded portion, and the camera 250 is integrated into a portion of the elongated support head 130 itself.

However, the camera 250 may also be mounted to protrude from a portion of the elongated support head 130. The camera 250 may also be adjustable, such that it is possible to extend and retract the camera 250 from the portion of the elongated support head 130, such as by having a camera 250 that pops out when in use.

In particular, FIG. 12 illustrates a video toothbrush where the camera 250 is mounted at the end of the elongated support head 130, and the bristles 120 form an opening generally beneath the camera 250 and are arranged partially surrounding the camera 250 on the cleaning head 110. In some embodiments, the opening may fully surround camera 250. In addition, embodiments are not limited to a single camera 250, as discussed above, and the camera(s) 250 may have different features and aspects. For example, there may be two cameras 250 providing a stereoscopic image. Alternatively, the camera 250 may include a macro lens or a fish-eye lens, as discussed above. The camera 250 may also include appropriate coatings, such as water-repellent coatings to improve image quality during tooth brushing.

The video toothbrush may also include an LED 614 as discussed earlier in the disclosure, such as an LED 614 mounted on the elongated support head 130 and configured to illuminate a field of view of the camera. Such an LED 614 may be a blue LED 614 or an ultraviolet LED 614. Furthermore, embodiments are not limited to a single LED 614, and the LED 614 may be mounted on or within other portions of the video toothbrush. For example, an LED 614 may be mounted within a casing of the camera 250 and adjacent to a lens of the camera 250. Such an LED 614 may also draw on a battery 612 as an energy source.

In general, the bristle portion 120 includes a plurality of bristles 120 arranged to expose an opening on the cleaning head 110, the opening providing the camera 250 with a view in a first direction. Generally, such a first direction is a direction perpendicular to the surface of the end of the cleaning head 110 and the surface of the end of the elongated support head 130, parallel to the bristles 120.

As noted above, in the example of FIG. 12, the bristles 120 are arranged such that the opening is at a top portion of the cleaning head 110. However, in an example where the camera 250 extends outward from a top portion of the elongated support head 130, it may be possible to cover the entirety of the cleaning head 110 with bristles 120, in that covering the entire cleaning head 110 with bristles 120 will not impede the operation of the camera 250. As discussed further, below, the bristles 120 may be of varying lengths, materials, and density. The bristles 120 may protrude outwards in a straight direction from the cleaning head 110 or may be inclined.

With respect to the cleaning head 110 and the elongated support head 130, each of these elements may have a flat surface opposite to each other and separated from each other by a first clearance 140. Moreover, the base of cleaning head 110 may be separated from a base portion 1223 of the elongated support head 130 by a second clearance 150. Also, as shown in FIG. 12 and discussed further, above, there may be an intervening portion of the elongated support head 130 between the handle 170 and the base of the cleaning head 110. In some embodiments, the first clearance 140 may be less than 5 mm, for example, about 4 mm, 3 mm, 2 mm, 1 mm, or less than 1 mm. In some embodiments, the second clearance 150 may be less than 5 mm, for example, about 4 mm, 3 mm, 2 mm, 1 mm, or less than 1 mm.

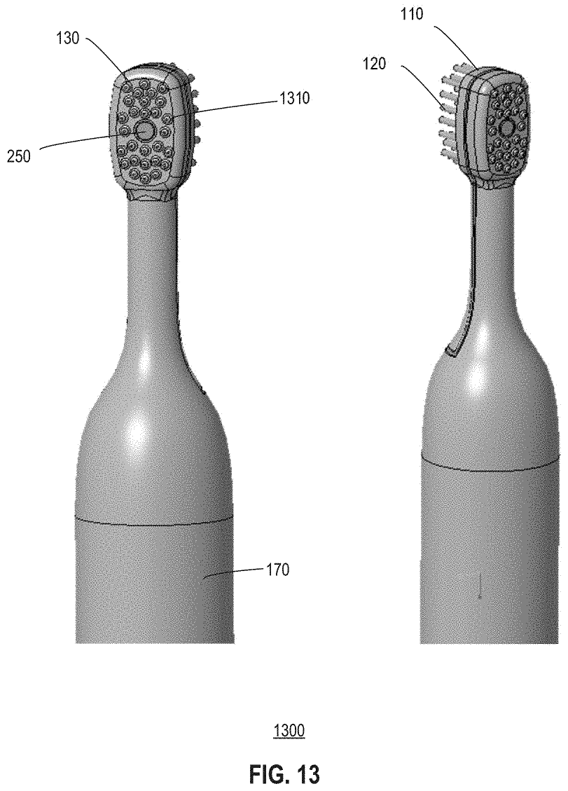

FIG. 13 illustrates perspective views of another video toothbrush 1300, according to certain embodiments. In FIG. 13, the camera 250 is arranged in a center of an elongated support head 130, which is generally similar to that of FIG. 12. However, a distinguishing aspect of FIG. 13 is that the camera 250 is embedded in the elongated support head 130 but is a rear-facing camera 250.

Thus, in FIG. 13, video toothbrush 1300 includes the camera 250, but on a rear surface of the elongated support head 130. Video toothbrush 1300 may be used to provide videos/images of a view in an opposite direction than the direction along which bristles 120 extends from the cleaning head 110.

FIG. 13 also illustrates the presence of bumps 1310 surrounding the camera 250. These bumps 1310 act to help clean the surface of the tongue. For example, the bumps 1310 may be made out of a plastic or a rubber material. The bumps 1310 may be made of a same material used for the elongated support head 130, in which case providing the bumps 1310 simply involves shaping a rear surface of the elongated support head 130 so that its surface is uneven rather than smooth. The bumps 1310 may be of even depth, or may be of varying depths, in various embodiments. However, in other embodiments, the bumps 1310 are formed of a material or materials different from that of the remainder of the elongated support head 130. For example, the bumps 1310 may be made of a rubber material, while the elongated support head 130 is made of a hard plastic.

However, these are merely examples, and it will be recognized that many other materials may be used for the elongated support head 130 and the bumps 1310, and these elements of the video toothbrush may include subparts made of different materials or different combinations of materials. In some embodiments, the main structure of the elongated support head 130 and the main structure of the cleaning head 110 are made of the same material, but these structures may be made of other materials in other embodiments. Additionally, it will be recognized that the bristles 120 may be made of a variety of materials or combinations of materials.

The bristles 120 may be soft bristles, medium bristles, hard bristles, or a combination of these types of bristles, and the bristles 120 may be of varying sizes. The cleaning head 110 may be detachable in certain embodiments, and the replacement cleaning heads 110 may be chosen to provide bristles 120 of varying hardnesses and sizes, as well as varying arrangements of bristles 120 based on where the camera 250 is situated.

Moreover, because camera 250 is provided to be mounted on a rear of the video toothbrush in FIG. 13, the camera 250 of FIG. 13 is able to provide images and/or video from a different perspective from the camera 250 placed as in FIG. 12, where the camera 250 is on the front side of the toothbrush. Specifically, because the camera 250 in FIG. 13 is positioned on a rear side of the video toothbrush, the camera 250 provides a perspective that better reflects what is seen from the opposite side of the bristles 120. In addition, because the camera 250 in FIG. 13 does not necessarily require an opening on the front of the video toothbrush, in the embodiments of FIG. 13, the entire surface of the cleaning head 110 may be covered with bristles 120.

However, because a camera 250 is present on the rear side of the elongated support head 130, the bumps 1310 on the rear side of the elongated support head 130 are arranged to provide an opening for camera 250 so that camera 250 is able to obtain its images and/or video. For example, the bumps may be arranged to surround the camera 250. It is noted that the various aspects of using a camera 250 discussed above with respect to FIG. 12 also apply, such as the use of special types of lenses, multiple cameras 250, lens coatings, LED elements) 614, and so on.

Additionally, FIG. 13 is not to be limited to an example where there is only a rear-facing camera 250, or only a rear-facing camera 250 that is positioned in a center of the rear side of the elongated support head 130. For example, in some embodiments, there are one or more front-facing camera(s) 250 and one or more rear-facing camera(s) 250. However, in such embodiments, it is desirable to have the bristles 120 and the bumps 1310 be arranged in a way such that the bristles 120 provide an opening for a front-facing camera 250 and the bumps 1310 provide an opening for a rear-facing camera 250.

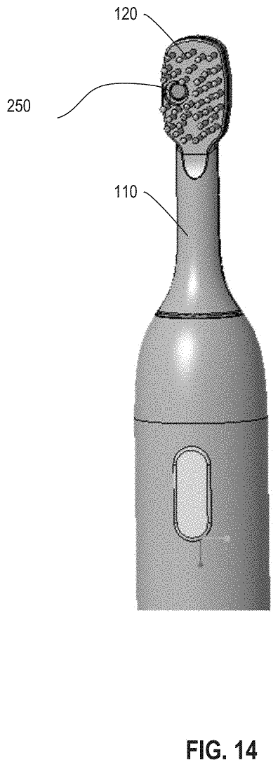

FIG. 14 illustrates a perspective view of a video toothbrush, according to certain embodiments. The embodiment of FIG. 14 is similar to that of FIG. 12, and duplicate explanation is omitted for brevity. However, in FIG. 14 the camera 250 is positioned on a side of the bristles 120, that is, a left side of the cleaning head 110 when facing a front of the cleaning head 110. Because the camera 250 is positioned to a left side of the elongated support head 130, as shown, it is desirable that the cleaning head 110 be designed to have an opening that corresponds to the shifted position of the camera 250.

FIG. 14 may be adapted for examples in which having the camera 250 be positioned to one side is desirable. For example, having the camera 250 be positioned to one side may provide better camera imagery or may increase the brushing effectiveness if one side of a user's mouth requires extra brushing, or having the camera 250 on one side or the other may make it more comfortable or effective for a user who is left-handed or right-handed. While FIG. 14 shows an example where the camera 250 is positioned directly next to an edge of the cleaning head 110, it will be recognized that the camera 250 need not be positioned directly next to the side of the cleaning head 110, but may instead merely be positioned off-center with respect to the cleaning head 110, between the center of the cleaning head 110 and the side of the cleaning head 110.

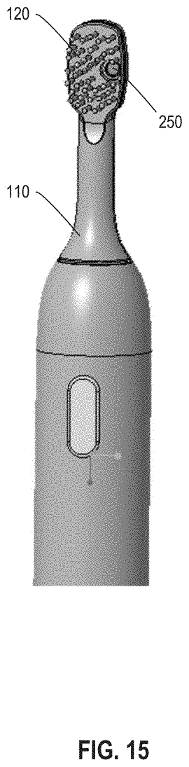

FIG. 15 illustrates a perspective view of a video toothbrush, according to certain embodiments. FIG. 15 illustrates an embodiment that is a counterpart of that of FIG. 14, where the camera 250 is positioned on the other side of the bristles 120, that is, a right side of the cleaning head 110 when facing a front of the cleaning head 110. Thus, in general, FIG. 15 is simply a mirror image of the video toothbrush of FIG. 14, given that the camera 250 is positioned on the other side of the cleaning head 110. As noted with respect to FIG. 14, in FIG. 15, the camera 250 need not be completely next to an edge of the cleaning head 110 but may simply be off-center with respect to the cleaning head 110. Also as noted in FIG. 14, FIG. 15 may facilitate better operation in certain use cases.

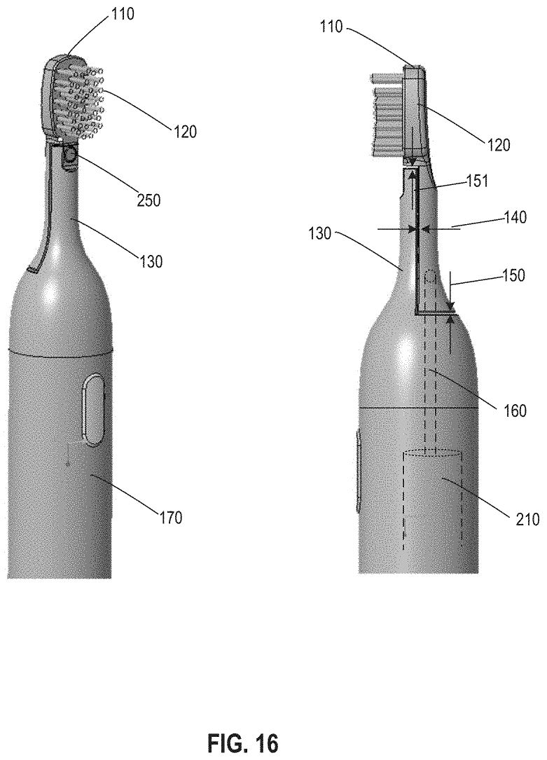

FIG. 16 illustrates perspective views of a video toothbrush, according to certain embodiments. While similar to the embodiments in FIG. 12, in FIG. 16, the camera 250 is positioned entirely beneath the bristles 120. Here, camera 250 is situated on a portion of the elongated support head 130 that is between the cleaning head 110 and the handle 170. For example, the cleaning head 110 may include a complete set of bristles 120.

FIG. 16 illustrates embodiments in which camera 250 is arranged to have a viewing angle perpendicular to the elongated support head 130. Additionally, in FIG. 16 there may be variants of camera 250, as discussed in conjunction with other embodiments as discussed above. For example, there may be multiple cameras 250, there may be LED(s) 614 that operate in conjunction with the camera(s) 250, the camera(s) 250 may have particular types of lenses, coatings, and so on.

As shown in FIG. 16, in addition to the first clearance 140 between the parallel fiat surfaces of elongated support head 130 and cleaning head 110 and the second clearance 150 between the base portion of elongated support head 130 and cleaning head 110, there is a third clearance 151 between the top of elongated support head 130 and the bristle portion 120 of cleaning head 110. The third clearance 151 may be less than 5 mm, such as 4 mm, 3 mm, 2 mm, 1 mm, or less than 1 mm.

Also, in FIG. 16, it is to be noted that at least two variant structures are possible in different embodiments. In one variation, the cleaning head 110 includes bristles 120, and is mounted on the elongated support head 130, entirely above the camera 250. In other embodiments, the cleaning head 110 is mounted on the distal end of the elongated support head 130 and includes a region with bristles 120, as well as a region that parallels part of the elongated support head 130 and includes a cutout region that provides a view region for the camera 250.

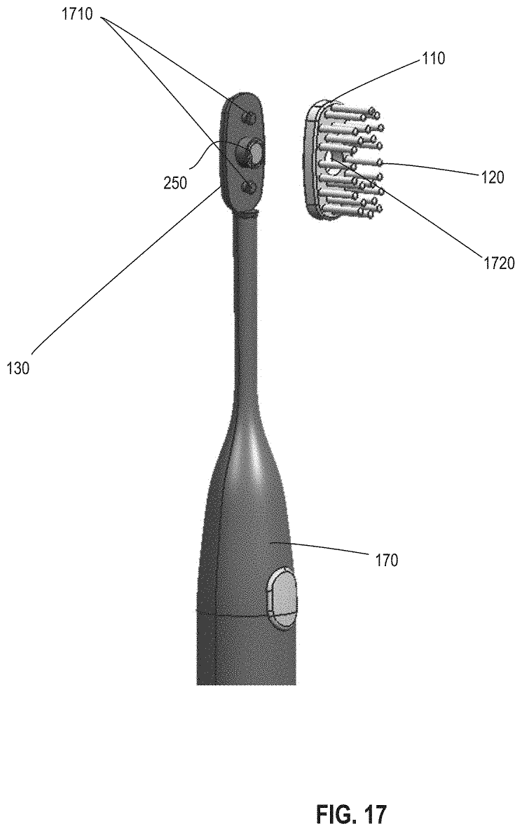

FIG. 17 illustrates a perspective view of a video toothbrush with a detachable cleaning head, according to certain embodiments. In FIG. 17, the detachable cleaning head 110 includes a plurality of bristles 120 surrounding an opening 1720. It is to be noted that in FIG. 17, the detachable cleaning head 110 only includes a base on which bristles 120 are attached, as well as an opening 1720 for the camera 250. The detachable cleaning head 110 of FIG. 17 does not include a neck, and the neck of the video toothbrush shown in FIG. 17 is entirely that of the elongated support head 130.

In FIG. 17, the camera 250 protrudes outward from the end portion of the elongated support head 130. The elongated support head 130 also includes other protrusions 1710 used to attach the cleaning head 110 to the elongated support head 130. While protrusions 1710 are illustrated as being round in FIG. 17, protrusions 1710 may take on other shapes, so long as they successfully match with the corresponding features of the cleaning head 110. Thus, when the cleaning head 110 is attached to the elongated support head 130, the bristles of the cleaning head 110 provide an opening 1720 and the camera 250 is able to capture visual information through the opening 1720. For example, FIG. 17 portrays opening 1720 as a round opening 1720, such that camera 250 protrudes through round opening 1720. However, it will be recognized that in other embodiments camera 250 and opening 1720 need not be round and may take on other shapes.

Moreover, in the example of FIG. 17, the cleaning head 110 has openings or indentations positioned at locations designed to engage with the protrusions 1710 to attach the cleaning head 110 to the elongated support member 130. For example, a rear side of the cleaning head 110 has openings or indentations that match with protrusions. By matching these portions of the cleaning head 110 with the protrusions 1710, the cleaning head 110 may be removably attached to the end of the elongated support head 130.

In FIG. 17, the figure illustrates a single camera 250, positioned in a center of the support head, as well as two protrusions 1710, one positioned above camera 250, and one positioned below camera 250. However, FIG. 17 is only an example, and there may be only a single protrusion 1710, or there may be more than two protrusions 1710. Also, the protrusions 1710 may be in different positions. For example, the protrusions 1710 may be positioned to each side of the camera 250, rather than being positioned above and below the camera 250.

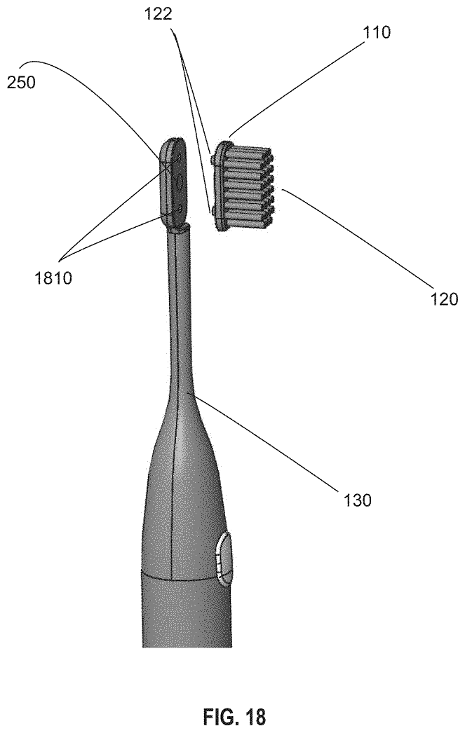

FIG. 18 illustrates a perspective view of a video toothbrush with a detachable cleaning head 110, according to certain embodiments. FIG. 18 illustrates an alternative detachable head to that of FIG. 17. In FIG. 18, the surface of camera 250 is level with or beneath the surface of the elongated support head 130. Moreover, in FIG. 18, the support head has indentations 1810. The detachable cleaning head 110 may include protrusions 122 corresponding to the indentations 1810, such that by matching the protrusions 122 with the indentations 1810, the detachable cleaning head 110 may be attached to the elongated support head 130. A similar description to that of FIG. 17 applies to FIG. 18 with respect to shared elements, and repeated description is omitted for brevity.

While FIG. 17 illustrates embodiments where the elongated support head 130 includes a protruding camera 250 as well as protrusions 1710 used to attach the cleaning head 110 to the elongated support head 130, and FIG. 18 illustrates embodiments where the elongated support head 130 includes a camera with a flat surface or a recessed surface and indentations used to receive protrusions of the cleaning head 110, it will be recognized that intermediate embodiments are possible. For example, the cleaning head 110 may include a combination of indentations 1810 and protrusions 1710 to attach it to the elongated support head 130.

Alternatively, the camera 250 itself may have a flat or recessed surface with respect to the surrounding portions of the elongated support head 130, but the camera 250 may also be surrounded by an edge region that protrudes and attaches the camera 250 to an indentation at the cleaning head 110. Additionally, as noted above, multiple cameras 250 may be present, some of which may protrude and some of which may have a flat or recessed surface. The camera 250 may also have the ability to change between a recessed, a flat, and a protruding status, for example, as a lens setting such as a focal depth changes, and the bristles 120 of the cleaning head 110 should be arranged to allow the camera 250 a clear view of what it needs to see.

While FIGS. 17-18 show embodiments where only the topmost portion of a cleaning head 110 is removable, embodiments are also possible in which a cleaning head 110 with a neck is removably attached to an elongated support head 130 that is structured to receive a cleaning head 110 with a neck, and these elements may have appropriate protrusions/indentations.

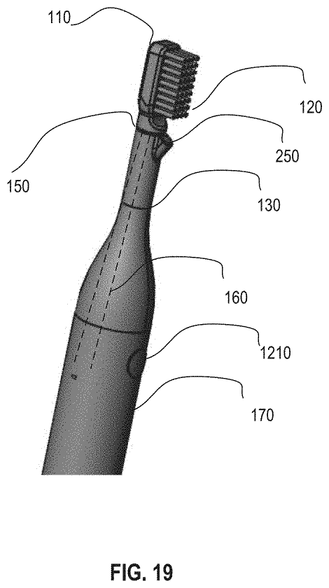

FIG. 19 illustrates a perspective view of a video toothbrush, according to certain embodiments. FIG. 19 shows embodiments that are somewhat different from the other embodiments, in that there is a cleaning head 110 with bristles 120 attached to an elongated support head 130, but the cleaning head 110 includes a complete set of bristles 120 covering a brushing surface, rather than a group of bristles 120 with an opening to allow a camera 250 to provide a view to capture images and/or video. FIG. 19 includes a camera 250 that is positioned at an end of the elongated support member 230, such as where the cleaning head 110 meets the elongated support head 130. In the other embodiments, generally, the camera 250 and the bristles 120 are parallel to one another and perpendicular to a surface of the cleaning head 110. However, in FIG. 19, there is a camera 250 that points out of the handle 170, and points towards the bristles 120 in a tilted direction.

Thus, the camera 250 provides somewhat different imagery than that of the other embodiments. While the other embodiments generally provide imagery from the perspective of the cleaning head, the camera 250 in FIG. 19 shows a camera 250 perspective that looks upward to where the brushing is occurring, rather than looking outward from a front or rear surface of the top of the video toothbrush.

FIG. 20 illustrates a perspective view of a video toothbrush and a paired display, according to certain embodiments. For example, FIG. 20 shows a video toothbrush 100, various aspects of which have been discussed, above. The video toothbrush 100 operates in a paired operation with a paired display 2010. For example, paired display 2010 may include a display unit 2020 and a sensor region 2030, but these are only example elements and paired display 2010 may include other elements in addition to these elements. For example, the display unit 2020 may be a flat panel display such as a liquid crystal diode (LCD) or light-emitting diode (LED) display, as examples.

For example, paired display 2010 may include a speaker to output sounds captured by a microphone that may be integrated into the video toothbrush 100 and/or a microphone that is integrated into the paired display 2010. For example, the paired display 2010 may permit interaction using voice commands or other forms of input that allow interaction with or control of the video toothbrush 100. The paired display 2010 may also include an element that wirelessly provides power to the video toothbrush 100, such as a wireless charger element for feeding energy to battery 612.

For example, the paired display 2010 may be fixed to a wall, or adjacent to or positioned on a mirror or a door, or above a sink, to facilitate real-time viewing of the images and/or video captured by the camera 250, such as during toothbrushing.

To permit interaction between the video toothbrush 100 and the paired display 2010, a number of features may be implemented. For example, the handle 170 of the video toothbrush 100 may include a wireless communication device, such as a device that uses Wi-Fi®, Bluetooth®, or another form of wireless communications that allows the video toothbrush 100 to send and receive data. For example, the video toothbrush 100 may include a communication module 620 that works in conjunction with a graphics processor 630 and is powered by a battery. In general, the communication structure presented in FIG. 20 corresponds to that previously discussed with respect to FIG. 6, except that additional aspects are discussed here.

For example, the embodiments of FIG. 20 may operate as follows. The video toothbrush 100 includes, as discussed above, camera(s) 250, a communication module 620, a motor 210, and a battery 612 as part of a power supply 610. The battery 612 and power supply 610 provide energy for the motor 210, the communication module 620, and the camera(s) 250, as needed. However, other embodiments are possible where the energy to operate various portions of the video toothbrush 100 is derived from another source, instead of or in addition to the battery 612. For example, power may be received over wires, transmitted wirelessly, generated from converting motion of the video toothbrush 100 into energy, or provided from a photovoltaic cell. The video toothbrush 100 may also include sensors that facilitate its ability to operate, as discussed further, below.

For example, the video toothbrush 100 may also include sensors that assist it in its ability to operate and provide useful information. The video toothbrush 100 may include, for example, motion sensors, distance sensors, or proximity sensors of different types. Such sensors may provide signals that indicate that the camera 250 and the communication module 620 should activate. The sensors may also activate the motion of the bristles 120, or may control the camera 250, such as by controlling a shutter, a lens, or lighting from an LED 614. As discussed above, the video toothbrush 100 may include a switch 1210. The video toothbrush 100 may include more than one switch 1210. The switch(es) 1210 may allow a user of the video toothbrush 100 to turn on and off the camera 250 and the communication module 620 if these are not activated automatically by a sensor. As discussed above, the switch(es) may be pressed or toggled to change between differing operational modes.

The paired display 2010 may also provide functionality that works in conjunction with the video toothbrush 100 to help a user take full advantage of the information gathered by the camera 250 at the video toothbrush 100. The paired display 2010 may have two operational states, a standby state, and a working state.

The paired display 2010 operates in a standby state when the video toothbrush 100 and the paired display 2010 are not in use. In the standby state, the paired display 2010 deactivates display unit 2020. However, sensor region 2030 is active, and may use sensors such as proximity sensors, motion sensors, and/or distance sensors built into the sensor region 2030 of the paired display 2010 to determine if a user is proximate to the paired display 2010. Once a proximity condition is met, which may involve factors such as a certain threshold duration of a certain threshold of proximity, the sensor region 2030 activates the paired display 2010 and the video toothbrush 100 to enter into the working state.

In the working state, the video toothbrush 100 and the paired display 2010 actively acquire images and/or video from the video toothbrush 100. Also in the working state, the communication module 620 of the video toothbrush 100 is active and communicates with a corresponding communication element of the paired display 2010. As discussed previously, such communication may use a local wireless protocol, such as Bluetooth® or Wi-Fi®, as examples. When the paired display 2010 enters into the working state, the paired display 2010 initiates a wireless connection with the video toothbrush 100. While the video toothbrush 100 operates in a standby state while not in use, the standby state should still allow the video toothbrush 100 to establish a wireless connection with the paired display 2010 so that the video toothbrush 100 can activate its communication module 620 when it receives a signal from the paired display 2010 to enter into the working state.

However, if the paired display 2010 attempts to enter into the working state because it satisfied the proximity criterion, but it cannot find a video toothbrush 100 with which to pair, the paired display 2010 may go back into the standby state. For example, the paired display 2010 may attempt for a five minute time period to establish a wireless connection with a video toothbrush 100, and reverts to the standby state if a successful connection has not been established within the five minute time period. It is to be noted that five minutes is merely an arbitrarily chosen example time interval, and other different time intervals may be used. It is also possible for the paired display 2010 to continue to try to establish a pairing relationship for an unlimited duration, but this approach may lead to wasted energy if the paired display 2010 detects motion but there is no video toothbrush 100 to connect to within a wireless range of the paired display 2010.

However, once the paired display 2010 has successfully established a wireless connection with the video toothbrush 100, the paired display 2010 and the video toothbrush 100 are able to exchange information. Specifically, the video toothbrush 100 transmits images and/or video captured by camera 250 to the paired display 2010, and the display unit 2020 displays the output of camera 250. The display unit 2020 may display video from the camera 250 in real time, or it may also potentially display images updated at regular intervals. In other embodiments, there may be a control such as a button on the video toothbrush 100, and when the button is pressed, it causes the camera 250 to update an image and/or captured by the video toothbrush 100. The paired display 2010 may also be capable of storing the images and/or video or sharing such images and/or video. For example, the paired display 2010 may be able to provide the images and/or video to a dentist to assist in the oral care of a user.

As discussed above, there may be multiple camera(s) 250 on the video toothbrush 100. Therefore, it is also possible that the paired display 2010 may simultaneously display multiple images and/or video provided by the video toothbrush 100 from multiple camera(s) 250. As noted, the multiple camera(s) 250 may provide a stereoscopic view from the perspective of the video toothbrush 100, and it is possible that the display unit 2020 is a three-dimensional (3D) display unit that provides a 3D image using a 3D display technology such as 3D glasses, in some embodiments.

Once the toothbrush is turned off, the paired display 2010 may enter standby mode again. For example, when the video toothbrush 100 is turned off, the paired display 2010 may enter into standby mode immediately or may wait for an interval such as five minutes to see if the user resumes using the video toothbrush 100 again before entering into standby. It may be desirable to wait for an interval before re-entering standby mode because it may consume resources or otherwise place a stress on the paired display to switch between standby and working mode, so it may be desirable to wait for a brief interval before re-entering standby mode in case the user is only briefly pausing his or her use of the video toothbrush 100.

Embodiments of the present disclosure may have various benefits and/or advantages. For example, certain embodiments may have a size and shape that are easy to use and have few impacts with teeth other than those being cleaned. Additionally, certain embodiments may have a profile that is smooth and consequently easy to comfortably insert into the user's mouth. Certain embodiments may provide a cleaning head that extends from the head and/or neck of the supporting head. This approach may permit a narrow gap between the cleaning head and the support head. For example, certain embodiments may be stably held against an inside surface of a user's cheek while brushing the outer surface of the teeth, particularly teeth such as molars near the back of the mouth. Certain embodiments may permit a natural adaptation between non-video toothbrush and video toothbrush by providing a form that is a close resemblance to a non-video toothbrush. Certain embodiments may provide a support head and neck that are structurally strong and configured to provide leverage that supports the action of the cleaning head.

Additional embodiments of the present disclosure are presented that provide additional variants of the technologies described herein that offer advantageous features. For example, additional embodiments have been provided that place the camera in additional positions and also provide different structures and arrangements for elements such as the cleaning head and the support head. Additionally, different approaches to using a removable cleaning head and attaching such a cleaning head have been discussed. Additional aspects of using a video toothbrush with a remote display have also been provided.

Another aspect of the disclosure is directed to a non-transitory computer-readable medium storing instructions which, when executed, cause one or more processors to perform the methods, as discussed above. The computer-readable medium may include volatile or non-volatile, magnetic, semiconductor, tape, optical, removable, non-removable, or other types of computer-readable medium or computer-readable storage devices. For example, the computer-readable medium may be the storage device or the memory module having the computer instructions stored thereon, as disclosed. In some embodiments, the computer-readable medium may be a disc or a flash drive having the computer instructions stored thereon.

It will be apparent to those skilled in the art that various modifications and variations can be made to the disclosed system and related methods. Other embodiments will be apparent to those skilled in the art from consideration of the specification and practice of the disclosed system and related methods.

It is intended that the specification and examples be considered as exemplary only, with a true scope being indicated by the following claims and their equivalents.

1. An apparatus, comprising:
a handle comprising an exciter;
an elongated support head extending from a proximal connection with the handle to a distal end, the elongated support head supporting a camera mounted near the distal end of the support head; and
a cleaning head extending in parallel with the elongated support head, the cleaning head comprising a bristle portion at a distal end of the cleaning head,
wherein:
the cleaning head is connected to the exciter by a shaft configured to transmit kinetic energy from the exciter to the cleaning head, at least a portion of the shaft being enclosed by the cleaning head; and
the bristle portion comprises a plurality of bristles arranged to expose an opening on the cleaning head, the opening providing the camera with a view in a first direction.

2. The apparatus of claim 1, wherein each of the cleaning head and the support head has a flat surface opposite to each other and separated from each other by a first clearance.

3. The apparatus of claim 1, wherein the cleaning head is separated from a base portion of the elongated support head by a second clearance.

4. The apparatus of claim 1, wherein the opening is arranged at a center of the cleaning head.

5. The apparatus of claim 1, wherein the opening is arranged at a left side or a right side of the cleaning head.

6. The apparatus of claim 1, wherein the opening is arranged at a top or a. bottom of the cleaning head.

7. The apparatus of claim 1, wherein the opening is surrounded by the plurality of bristles.

8. The apparatus of claim 1, wherein the plurality of bristles extend parallel to the first direction.

9. The apparatus of claim 1, wherein the cleaning head is removably attached to the shaft.

10. The apparatus of claim 1, further comprising:
a communication interface, wherein the communication interface is configured to communicate data between the camera and a remote device.

11. The apparatus of claim 1, wherein the cleaning head is configured to receive the kinetic energy in order to translate the plurality of bristles with respect to the opening.

12. The apparatus of claim 1, wherein the cleaning head is configured to receive the kinetic energy in order to rotate the plurality of bristles with respect to the opening.

13. An apparatus, comprising:
a handle comprising an exciter;
a support head extending from a proximal connection with the handle to a distal end of the support head, the support head supporting a camera and comprising mounting elements for a cleaning head having a plurality of bristles, the mounting elements being configured to removably mount the cleaning head to the support head near the distal end of the support head; and
a shaft extending from the exciter to the distal end of the support head, the shaft being configured to transmit kinetic energy from the exciter to the cleaning head through the mounting elements,
wherein the camera is configured to provide a view in a first direction through an opening of the cleaning head after the cleaning head is installed onto the support head through the mounting elements.

14. The apparatus of claim 13, wherein:
the camera protrudes from the support head and through the opening of the cleaning head; and
the support head comprises protrusions as the mounting elements that correspond to indentations on the mounting elements, the cleaning head being installed by attaching the protrusions to the indentations.

15. An apparatus, comprising:
a handle comprising an exciter;
a support head extending from a proximal connection with the handle to a distal end, the support head supporting a camera at the distal end of the support head; and
a shaft extending from the exciter to the distal end of the support head, the shaft being configured to support a cleaning head comprising a plurality of bristles at a distal end of the shaft and to transmit kinetic energy from the exciter to the cleaning head,
wherein the camera is positioned on the support head between the cleaning head and the handle.

16. The apparatus of claim 15, wherein the camera is oriented to be parallel to the plurality of bristles.

17. The apparatus of claim 15, wherein the camera is oriented to be tilted towards the plurality of bristles.

18.-20. (canceled)
